(19) United States
(12) Patent Application Publication
(10) Pub. No.: US 2026/0324959 A1
(43) Pub. Date: Oct. 22, 2026

(54) CYSTOSCOPY DEVICE

(71) Applicant: THE BOARD OF REGENTS OF THE UNIVERSITY OF TEXAS SYSTEM, Austin, TX (US)

(72) Inventors: Philippe E. Zimmern, Dallas, TX (US); Tatum Shipe, Dallas, TX (US); Sofia Mendez, Dallas, TX (US); Frank Parsley, Dallas, TX (US); Pedro Ruiz, Dallas, TX (US); Tofunmi Sodimu, Dallas, TX (US)

(21) Appl. No.: 19/104,421

(22) PCT Filed: Jul. 17, 2023
(86) PCT No.: PCT/US2023/070341

§ 371 Date: Feb. 18, 2025

Related U.S. Application Data
(60) Provisional application No. 63/374,989, filed on Sep. 8, 2022.

(51) Int. Cl. A61B 1/015; A61B 1/00; A61B 1/303; A61M 3/02

(52) U.S. Cl. CPC A61B 1/015; A61B 1/00119; A61B 1/303; A61M 3/0202; A61M 2205/3393; A61M 2205/583; A61M 2205/8206

(57) ABSTRACT

A cystoscopy device includes a volume measurement component operable to determine in real time an infused volume of an irrigating fluid from an intravenous (IV) bag that is infused during a cystoscopy procedure. A user interface component is in communication with the volume measurement component. The user interface component is operable to display the infused volume of the irrigating fluid to a user.

### CROSS-REFERENCE TO RELATED APPLICATIONS

This application claims the benefit of U.S. Provisional Patent Application Ser. No. 63/374,989 filed on Sep. 8, 2022, the contents of which are incorporated by reference in its entirety.

### BACKGROUND

### 1. Field

The present inventive concept is directed to cystoscopy devices operable to determine an infused volume of an irrigating fluid that is infused during a cystoscopy procedure.

### 2. Discussion of Related Art

During a cystoscopy procedure, a surgeon inserts a cystoscope—a tube that consists of a camera to view and record images—into the bladder through the urethra. During the cystoscopy procedure, irrigating fluid is needed to see inside the bladder. The irrigation fluid is instilled into the bladder to enable inspection of a surface of the bladder via an instillation process. There is a connection on the side of the cystoscope to an intravenous (IV) tubing which is connected to a fluid bag (e.g., an IV bag) containing either saline, water, or glycine in quantities up to six (6) liters (L). Cystoscopies can be diagnostic or therapeutic in nature. The doctor may record or photograph the images for diagnostic or monitoring purposes (clinical) and/or for comparison of similar conditions in the same patient over time (clinical) and/or across patients (clinical/research).

### BRIEF SUMMARY

The present inventive concept provides a cystoscopy device operable to determine the amount of irrigating fluid infused or instilled into a patient's bladder during a cystoscopy procedure. The cystoscopy device may include a mass sensor operable to determine a mass of an IV bag. A controller coupled with the mass sensor may be operable to convert the change of the mass of the IV bag to volume change of the irrigating fluid in the IV bag. The volume change may correspond with the infused volume of the irrigating fluid infused during the cystoscopy procedure.

The aforementioned may be achieved in one aspect of the present inventive concept by a cystoscopy device including a volume measurement component and a user interface component. The volume measurement component may be operable to determine in real time an infused volume of an irrigating fluid from an intravenous (IV) bag that is infused during a cystoscopy procedure. The user interface component may be in communication with the volume measurement component. The user interface component may be operable to display the infused volume of the irrigating fluid to a user.

The volume measurement component may include a mass sensor operable to determine a mass of the IV bag. The mass sensor may include a load cell. The IV bag may be suspended from the load cell. A controller may be coupled with the mass sensor. The controller may be operable to determine a change in mass of the IV bag and convert the change in mass to a change in volume of the irrigating fluid in the IV bag. The change in volume of the irrigating fluid in the IV bag may correspond with the infused volume of the irrigating fluid. The cystoscopy device may be configured to be retrofit to a conventional cystoscope and a conventional IV bag.

The volume measurement apparatus may include a mass sensor operable to determine a change in mass of the IV bag, and a controller coupled with the mass sensor. The controller may be operable to convert the change in mass to a change in volume of the irrigating fluid in the IV bag. The change in volume of the irrigating fluid in the IV bag may correspond with the infused volume of the irrigating fluid.

The infused volume may be displayed in real time. The user interface component may be sterilizable. The user interface component may be operable to be coupled with IV tubing that fluidly coupled the IV bag with a cystoscope. The volume measurement component and/or the user interface component may be powered by a battery.

The aforementioned may also be achieved in one aspect of the present inventive concept by a cystoscopy system including a cystoscope, and intravenous (IV) bag containing irrigating fluid, and a cystoscopy device. The IV bag may be fluidly coupled with the cystoscope via an IV tubing such that the irrigating fluid is instilled inside a bladder of a patient during a cystoscopy procedure. The cystoscopy device may include a volume measurement component operable to determine in real time an infused volume of the irrigating fluid from the IV bag that is infused during the cystoscopy procedure, and a user interface component in communication with the volume measurement component. The user interface component may be operable to display the infused volume of the irrigating fluid to a user.

The aforementioned may also be achieved in one aspect of the present inventive concept by a method including determining, by a mass sensor, a change in mass of an intravenous (IV) bag as irrigating fluid is infused into a bladder during a cystoscopy procedure. A controller may convert the change in mass to a change in volume of the irrigating fluid in the IV bag. The change in volume of the irrigating fluid in the IV bag may correspond with the infused volume of the irrigating fluid. The infused volume of the irrigating fluid may be displayed in real time by a user interface component to a user.

Additional aspects, advantages, and utilities of the present inventive concept will be set forth, in part, in the description which follows and, in part, will be obvious from the description, or may be learned by practice of the present inventive concept.

The foregoing is intended to be illustrative and is not meant in a limiting sense. Many features and subcombinations of the present inventive concept may be made and will be readily evident upon a study of the following specification and accompanying drawings comprising a part thereof. These features and subcombinations may be employed without reference to other features and subcombinations.

### BRIEF DESCRIPTION OF THE DRAWINGS

The description will be more fully understood with reference to the following figures and data graphs, which are presented as various embodiments of the present inventive concept and should not be construed as a complete recitation of the scope of the present inventive concept, wherein:

FIG. 1 is a perspective view of a cystoscopy system, in accordance with embodiments of the present inventive concept;

FIG. 2 is a partial view of the cystoscopy system with a wall for a volume measurement component omitted, in accordance with embodiments of the present inventive concept;

FIG. 3 is a perspective view of a volume measurement component with a wall omitted, in accordance with embodiments of the present inventive concept;

FIG. 4 is a schematic diagram of a cystoscopy device, in accordance with embodiments of the present inventive concept;

FIG. 5 is a schematic diagram of a controller, in accordance with embodiments of the present inventive concept;

FIG. 6 is a flow chart illustrating steps for operation of a cystoscopy device, in accordance with embodiments of the present inventive concept; and

FIG. 7 is a flow chart illustrating steps for a method to utilize a cystoscopy device, in accordance with embodiments of the present inventive concept.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The illustrations and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

### I. terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

### II. General Architecture

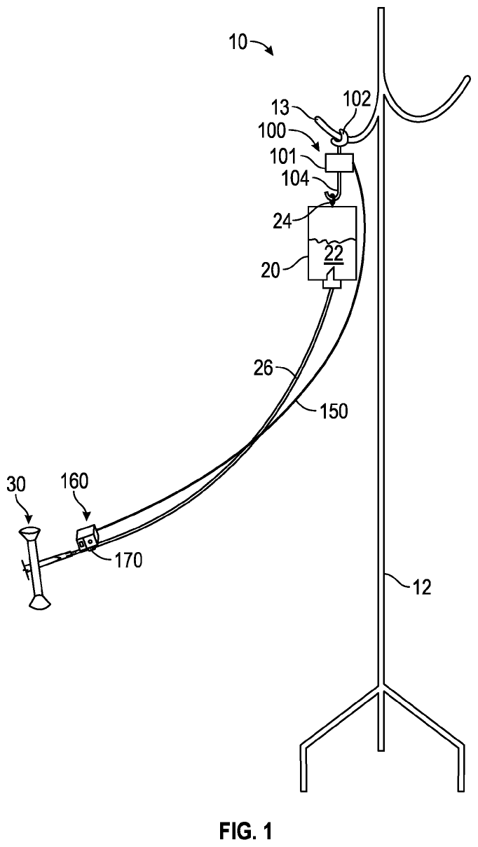

Turning to FIG. 1, a cystoscopy system 10 to perform cystoscopy procedures is provided. The cystoscopy system 10 can include a cystoscope 30, and intravenous (IV) bag 20, and a cystoscopy device 100. The cystoscope 30 can be operable to be inserted into a patient to look inside the bladder of the patient. The cystoscope 30 can include a camera to provide visual access to the inside of the bladder. In at least one example, the cystoscope 30 can be operable to be inserted into the bladder of the patient, for example, via a urethra of the patient. Other means of accessing the bladder of the patient are foreseen and within the scope of the present inventive concept. For example, the bladder of the patient may be accessed via a suprapubic tract, which may be preferred if, for example, the urethra of the patient is closed or strictured. Another foreseen alternative of the present inventive concept is to access a neobladder or a continent reservoir of the patient, for example, via a cutaneous stoma, which may be placed on a right and/or left side and/or at the umbilicus of the patient.

To provide a better view of the bladder wall of the patient, irrigating fluid 22 can be instilled inside the bladder of the patient during the cystoscopy procedure. The IV bag 20 can be operable to contain the irrigating fluid 22. In at least one example, the irrigating fluid 22 can include saline, glycine, water, and/or other suitable irrigating fluid.

The IV bag 20 can be fluidly coupled with the cystoscope 30 via an IV tubing 26 such that the irrigating fluid 22 is instilled or infused inside the bladder of the patient during the cystoscopy procedure. Accordingly, the irrigating fluid 22 can flow from the IV bag 20 through the IV tubing 26 to the bladder of the patient, in some examples via the cystoscope 30.

The cystoscopy device 100 can be operable to determine the instilled or infused volume of the irrigating fluid 22 from the IV bag 20 that is infused during the cystoscopy procedure. Cystoscopy procedures can be diagnostic or therapeutic in nature. The user (e.g., doctor, physician's assistant, nurse practitioner, etc.) may record or photograph the bladder of the patient for diagnostic purposes, monitoring purposes, and/or research purposes for comparison of similar conditions across patients. By being able to measure, track, and record the infused volume of the irrigating fluid 22 that is infused in the bladder at the time of the recording and/or photograph, the user is able to better correlate and compare bladder wall findings having the same infused volume of the irrigating fluid 22. Knowing the infused volume of irrigating fluid 22 at a correlating time of the recording and/or photograph enables users (e.g., doctors, researchers, etc.) to take images of the bladder and/or study the bladder at different infused volumes, allowing them to accurately set standards for width, spacing, and/or number of bladder wall trabeculations at certain infused volumes in patients of a certain size, age, and/or gender.

Conventional methods used for the measurement of irrigating fluid in the bladder rely on qualitative observations from the user, as well as external measurements upon drainage of the irrigating fluid into a graduated container. This can provide inaccurate, inconsistent, inefficient, and unreliable results. For example, during conventional cystoscopy procedures, the operator surgeon can use one of the following three methods to determine degree of bladder fill: observation, visual grading of bladder wall findings (for example, trabeculations, cystitis involvement grading, etc.), or external measurement of bladder fill volume.

With observation, the surgeon visually looks for dripping or leakage around the cystoscope when the bladder has reached its full capacity to determine the volume at which maximum bladder capacity was reached under anesthesia. This conventional method is rather subjective, and is unable to give specific measurements—only the maximum volume to fill a particular bladder. With visual grading, the patient's bladder wall is graded from I to IV based on the relative thickness of the bladder wall. This method does not consider the variance in bladder fill when comparing one patient to the next. Visual grading is inconsistent between doctors since the grading is done at different bladder volumes unless standardized by a pre-established research protocol, which has not been done due to being very tedious. Finally, external measurement cannot be performed until completion of the cystoscopy procedure. With external measurement, the bladder is drained once the maximum bladder capacity has been reached and the volume drained is measured in a separate container or pitcher with graduations every 50 or 100 milliliter (mL) depending on the pitcher graduation scale. This practice is time consuming and inconvenient to perform, especially during surgery.

Accordingly, with the cystoscopy device 100, the cystoscopy procedure can be safer and more efficient. During the cystoscopy procedure, the user (e.g., physician, physician's assistant, nurse practitioner, etc.) can determine how much irrigating fluid 22 is infused within the bladder of the patient at any given time, and the user can perform cystoscopy procedures under better control of actual bladder volume and gain access to standardized studies of bladder wall findings (for example trabeculations, cystitis involvement grading, etc.) when the bladder's infused volume is constant across all study participants and/or patients.

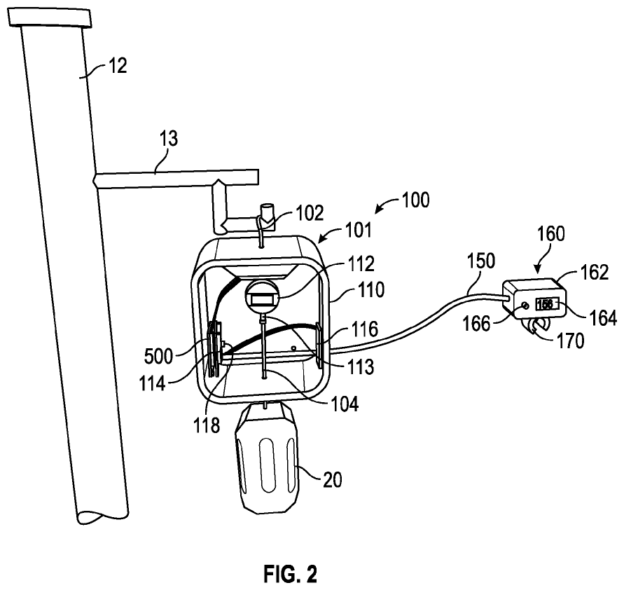

Referring to FIGS. 1 and 2, the cystoscopy device 100 can include a volume measurement component 101 and a user interface component 160. The volume measurement component 101 can be operable to determine in real time an infused volume of the irrigating fluid 22 from the IV bag 20 that is infused into the patient's bladder during the cystoscopy procedure.

In at least one example, the IV bag 20 can be suspended from the volume measurement component 101. For example, in some examples, the volume measurement component 101 can include a catch 104 operable to receive a link 24 of the IV bag 20. The catch 104 can be received through the link 24 of the IV bag 20 such that the IV bag 20 is coupled with the catch 104. In some examples, as illustrated in FIG. 2, the catch 104 can be a portion of the IV bag 20.

In at least one example, the cystoscopy device 100 (e.g., the volume measurement component 101) can be hung from an arm 13 of a stand 12, for example via a hook 102. In other examples, the cystoscopy device 100 can be coupled with the stand 12 via other mechanisms without deviating from the scope of the disclosure. The cystoscopy device 100 can be configured so that the cystoscopy device 100 is hung and/or coupled with the stand 12 in the same way that the IV bag 20 would hang and/or be coupled with the stand 12 if the cystoscopy device 100 was not being utilized. Accordingly, the cystoscopy device 100 does not require any changes or modifications to conventional IV bags 20 and can be retrofit to a conventional IV bag 20.

The user interface component 160 can be in communication with the volume measurement component 101, and the user interface 160 can be operable to display the infused volume of the irrigating fluid 22 to the user. In some examples, the infused volume can be displayed to the user in real time. Accordingly, the user can know how much infused volume has been infused into the bladder of the patient and can control the cystoscope 30 and the IV bag 20 accordingly (e.g., take a picture, pause and/or stop further flow of the irrigating fluid 22 from the IV bag 20 into the bladder, etc.). As illustrated in FIG. 1, the user interface component 160 can be in communication with the volume measurement component 101 via a wired connection 150. With the wired connection, the user interface component 160 can receive power and/or transmit data back to the volume measurement component 101. In some examples, the user interface component 160 can be in communication with the volume measurement component 101 via a wireless connection, for example via Bluetooth, WiFi, etc. In such an example, the user interface component 160 may be powered via battery.

As illustrated in FIG. 1, the user interface component 160 can be coupled with the IV tubing 26 near the cystoscope 30 via a fastener 170. The fastener 170 may include an adhesive cable holder, a hook and loop fastener, an elastic band, and/or any other suitable mechanism to couple the user interface component 160 with the IV tubing 26 without deviating from the scope of the disclosure. By having the user interface component 160 coupled with the IV tubing 26 near the cystoscope 30, the user can refer to the user interface component 160 easily without needing to avert their eyes far away from the cystoscope 30 and the patient. In some examples, the user interface component 160 being coupled to the IV tubing 26 away from the volume measurement component 101 allows for only requiring the user interface component 160 to be sterilizable. Accordingly, for a new cystoscopy procedure, the user interface component 160 can be detached and/or replaced while the same volume measurement component 101 can be reused. In some examples, both of the user interface component 160 and the volume measurement component 101 can be sterilizable. In some examples, the user interface component 160 may be disposed in and/or covered by a sterile sheath when the used in the operating room. The sheath can have at least a portion that is at least partially transparent such that the user can see the display 164 through the sheath. In some examples, for example in an office setting, there may not be a need to sterilize the user interface component 160 since the user interface component 160 may be positioned away from the prepped perineum area.

The configuration of the volume measurement component 101 and the user interface component 160 allows for the cystoscopy device 100 to be retrofit to a conventional cystoscope and a conventional IV bag. Additionally, the cystoscopy device 100 does not come in direct contact with any sterile fluid and is non-invasive, which limits the necessity of sterilization of the cystoscopy device 100. Accordingly, the cystoscopy device 100 is non-invasive and minimizes risk of infection and/or complications for the patient during the cystoscopy procedure.

Referring to FIGS. 2, 3, and 4, the volume measurement component 101 can include a housing 110 which can include the hook 102 to couple the housing 110 to the stand 12. In at least one example, the housing 110 can include a junction box. The housing 110 can be strong, lightweight, impact resistant, and/or be able to be sanitized (e.g., disinfecting and/or sterilizing) with ease. In at least one example, the housing 110 can be tested using NEMA ratings and be drop resistant, water-resistant, waterproof, and/or corrosive resistant. In at least one example, the housing 110 can be made of polycarbonate material which can be strong and can withstand up to 900 inch-pounds of force while being lightweight and nonporous for sanitizing. Accordingly, the housing 110 can be suitable for clinical or surgical settings.

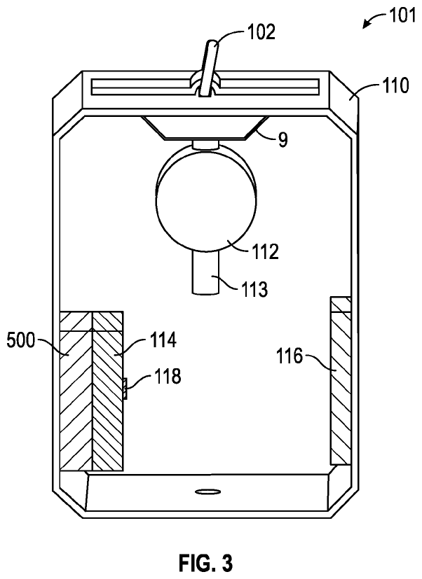

Disposed in the housing 110, the volume measurement component 101 can include a mass sensor 112. In at least one example, the mass sensor 112 can include a load cell. The mass sensor 112 can be operable to determine a mass of the IV bag 20. For example, the IV bag 20 can be suspended from the load cell 112, and as the irrigating fluid 22 leaves the IV bag 20 to be infused in the bladder of the patient, the load cell 112 can measure the adjusted mass of the IV bag 20. In at least one example, as illustrated in FIGS. 2 and 3, the load cell 112 can be suspended from and/or coupled with the housing 110 via a bracket 9. In at least one example, as illustrated in FIGS. 2 and 3, the load cell 112 can include a coupling component 113 from which a catch 104 can extend. The IV bag 20 can be coupled with the catch 104 and be suspended from the load cell 112.

The load cell 112 can be operable to measure tension and/or compression. When the load cell 112 is loaded (e.g., coupled with the suspended IV bag 20), the value of a variable resistor changes, inducing a voltage across the output terminals of the load cell 112. This voltage can be directly proportional to the applied force and can be used to determine the mass of the IV bag 20.

A controller 500 can be coupled with the mass sensor 112. The controller 500 can be operable to receive the measurements of the mass of the IV bag 20 from the mass sensor 112. With the measurements of the mass of the IV bag 20, as the irrigating fluid 22 flows out of the IV bag 20, the controller 500 can be operable to determine a change in mass of the IV bag 20. In some examples, the controller 500 can determine the change in mass of the IV bag 20 in real time. With the change in mass of the IV bag 20, the controller 500 can be operable to convert the change in mass to a change in volume of the irrigating fluid 22 in the IV bag 20. Because the mass being lost from the IV bag 20 is only going to the bladder of the patient, the mass lost is directly proportional to the amount that is being infused into the bladder. Accordingly, the change in volume of the irrigating fluid in the IV bag 20 corresponds with the infused volume of the irrigating fluid 22 in the bladder.

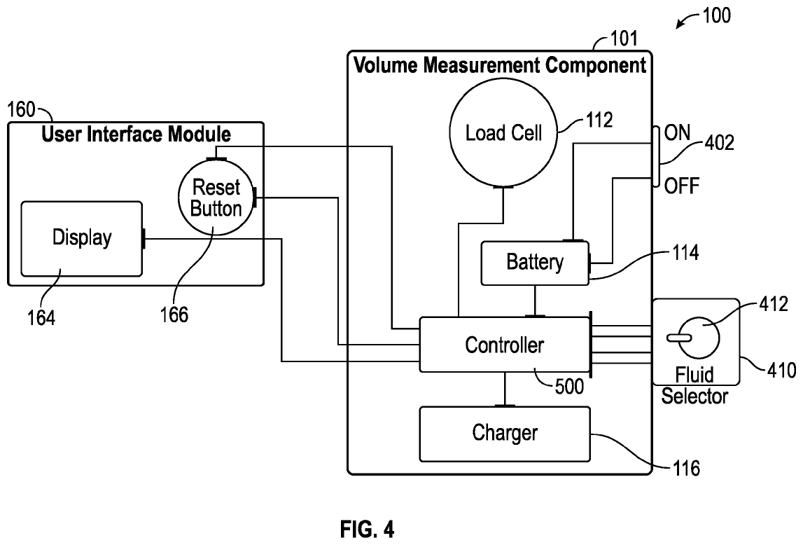

The controller 500 can convert the mass of the IV bag 20 to the infused volume of the irrigating fluid 22 infused into the bladder beginning with input of the type of irrigating fluid 22 being utilized. For example, as illustrated in FIG. 4, the volume measurement component 101 can include a fluid selector 410. The fluid selector 410 can include a switch 412 which can be used to input the irrigating fluid 22 being utilized in the cystoscopy procedure. For example, the volume measurement component 101 can be calibrated and programmed for at least three different irrigating fluids 22, for example saline, glycine, and water. Each of saline, glycine, and water may have different densities. Accordingly, by inputting the type of irrigating fluid 22 being utilized, the controller 500 can perform the correct calculations to convert mass to volume based on the density of the irrigating fluid 22 being utilized.

In at least one example, the initial volume of irrigating fluid 22 in the IV bag 20 before the cystoscopy procedure begins can be input. Accordingly, the controller 500 can subtract the volume of irrigating fluid 22 in the IV bag 20 from the initial volume of irrigating fluid 22 to determine the infused volume of irrigating fluid 22 that is infused in the bladder of the patient. In some examples, the initial volume of irrigating fluid 22 may not need to be input if the initial volume is standardized and consistent between IV bags 20.

In at least one example, the controller 500 can be coupled with the cystoscope 30 and/or a processor can be coupled with both the controller 500 and the cystoscope 30. The controller 500 and/or the processor can utilize the calculated infused volume to directly correlate the images and/or videos taken by the cystoscope 30 with the infused volume at the time of the image and/or video.

In at least one example, the volume measurement component 101 can be powered via a battery 114. In some examples, the battery 114 can be coupled with the controller 500 to provide power to the controller 500. As an average time for a single cystoscopy procedure can be about 20 minutes, the life of the battery 114 can exceed 20 minutes. In some examples, the life of the battery 20 can be at least 60 minutes so that the cystoscopy device 100 can be utilized for multiple procedures without requiring charging, for example via charger 116. In some examples, the volume measurement component 101 can be powered while being plugged in, by having a battery 114, the volume measurement component 101 can be easily movable, and the cystoscopy device 100 does not provide more clutter via more wires and cables. In some examples, the battery 114 can be replaceable so that new and fully charged batteries can replace a depleted battery.

As illustrated in FIG. 4, a power switch 402 can be coupled with the battery 114. As the power switch 402 is turned to “on”, the battery 114 may provide power, and when turned to “off”, the battery 114 may be prevented from providing power. This can conserve power and extend the life of the battery 114.

In at least one example, the volume measurement component 101 can include an amplifier 118. In some examples, the output voltage of the load cell 112 can be small. Accordingly, it may be necessary to amplify and filter the signal from the load cell 112 before passing it to the controller 500 for processing.

Referring to FIGS. 1, 2, and 4, the user interface module 160 an include a display 164 operable to display the infused volume of the irrigating fluid 22 to the user. In some examples, the infused volume can be displayed in real time. In at least one example, the display 164 may be backlit such that the user can read the display in a darkened room during the cystoscopy procedure.

In at least one example, the user interface module 160 can include a reset button 166. The reset button 166 can be pushed to reinitialize the current mass of the IV bag 22 as the initial mass, thus zeroing the volume. When the reset button 166 is pushed, the controller 500 can be signaled to restart calculations of irrigating fluid 22 infused through the bladder of the patient. In some examples, the reset button 166 can be utilized to track intervals, for example every 100 milliliters.

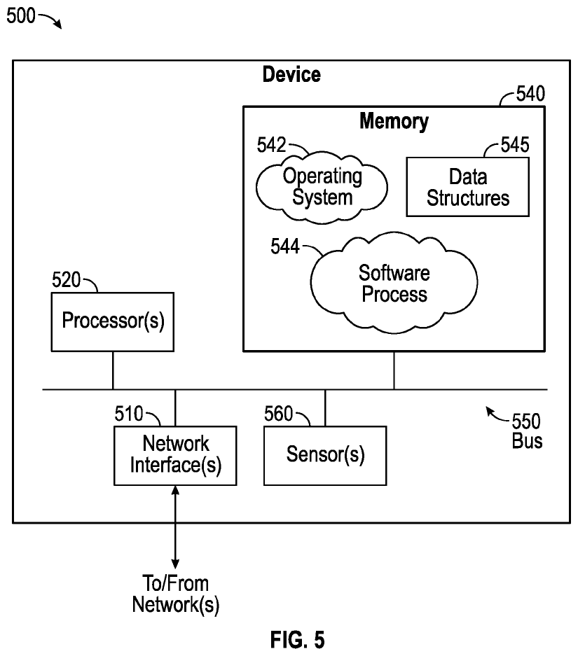

FIG. 5 is a block diagram of an exemplary controller 500. Controller 500 is configured to perform processing of data and communicate with the mass sensor, for example as illustrated in FIGS. 1-4. In operation, controller 500 communicates with one or more of the above-discussed components and may also be configured to communication with remote devices/systems.

As shown, controller 500 includes hardware and software components such as network interfaces 510, at least one processor 520, sensors 560 and a memory 540 interconnected by a system bus 550. Network interface(s) 510 can include mechanical, electrical, and signaling circuitry for communicating data over communication links, which may include wired or wireless communication links. Network interfaces 510 are configured to transmit and/or receive data using any variety of different communication protocols.

Processor 520 represents a digital signal processor (e.g., a microprocessor, a microcontroller, or a fixed-logic processor, etc.) configured to execute instructions or logic to perform tasks in a wellbore environment. Processor 520 may include a general purpose processor, special-purpose processor (where software instructions are incorporated into the processor), a state machine, application specific integrated circuit (ASIC), a programmable gate array (PGA) including a field PGA, an individual component, a distributed group of processors, and the like. Processor 520 typically operates in conjunction with shared or dedicated hardware, including but not limited to, hardware capable of executing software and hardware. For example, processor 520 may include elements or logic adapted to execute software programs and manipulate data structures 545, which may reside in memory 540.

Sensors 560, which may include the mass sensor as disclosed herein, typically operate in conjunction with processor 520 to perform measurements, and can include special-purpose processors, detectors, transmitters, receivers, and the like.

Memory 540 comprises a plurality of storage locations that are addressable by processor 520 for storing software programs and data structures 545 associated with the embodiments described herein. An operating system 542, portions of which may be typically resident in memory 540 and executed by processor 520, functionally organizes the device by, inter alia, invoking operations in support of software processes and/or services 544 executing on controller 500. These software processes and/or services 544 may perform processing of data and communication with controller 500, as described herein. Note that while process/service 544 is shown in centralized memory 540, some examples provide for these processes/services to be operated in a distributed computing network.

Other processor and memory types, including various computer-readable media, may be used to store and execute program instructions pertaining to the fluidic channel evaluation techniques described herein. Also, while the description illustrates various processes, it is expressly contemplated that various processes may be embodied as modules having portions of the process/service 544 encoded thereon. In this fashion, the program modules may be encoded in one or more tangible computer readable storage media for execution, such as with fixed logic or programmable logic (e.g., software/computer instructions executed by a processor, and any processor may be a programmable processor, programmable digital logic such as field programmable gate arrays or an ASIC that comprises fixed digital logic. In general, any process logic may be embodied in processor 520 or computer readable medium encoded with instructions for execution by processor 520 that, when executed by the processor, are operable to cause the processor to perform the functions described herein.

Additionally, the controller 500 can apply machine learning, such as a neural network or sequential logistic regression and the like, to determine relationships between the reflected signals from the pressure pulses received by the sensors 130. For example, a deep neural network may be trained in advance to capture the complex relationship between the mass of an IV bag and the volume of irrigating fluid being infused into the bladder during the cystoscopy procedure.

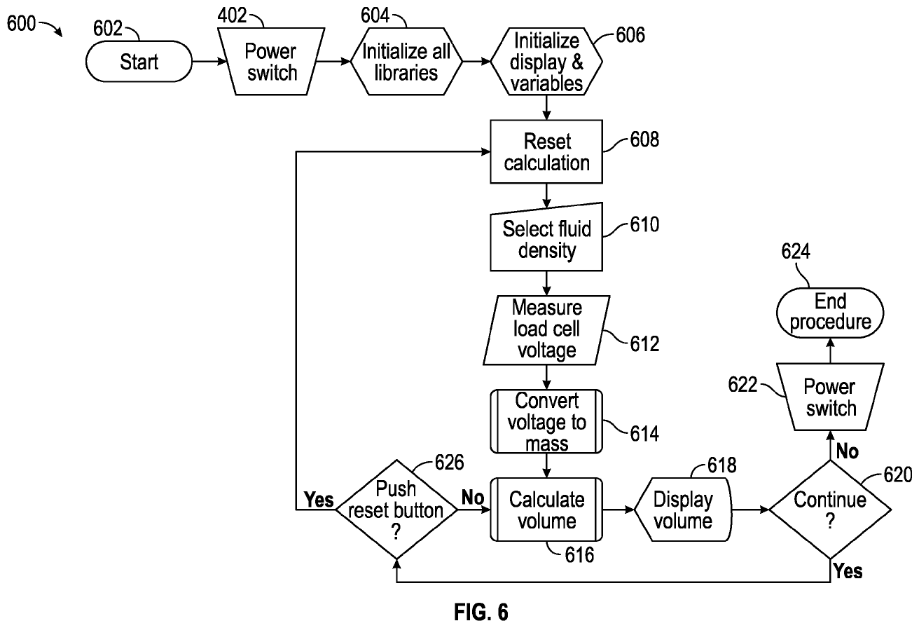

Referring to FIG. 6, a flowchart is presented in accordance with an example embodiment. The method 600 is provided by way of example, as there are a variety of ways to carry out the method. The method 600 described below can be carried out using the configurations illustrated in FIG. 1-5, for example, and various elements of these figures are referenced in explaining example method 600. Each block shown in FIG. 6 represents one or more processes, methods or subroutines, carried out in the example method 600. Furthermore, the illustrated order of blocks is illustrative only and the order of the blocks can change according to the present disclosure. Additional blocks may be added or fewer blocks may be utilized, without departing from this disclosure. The example method 600 can begin at block 602.

At block 602, the method begins by turning on the power switch 402. At block 602, all libraries are initialized. At block 606, the display 164 on the user interface component 160 and the variables are initialized. At block 608, the calculation for infused volume is reset. At block 610, the fluid density of the irrigating fluid 22 is selected. In at least one example, the fluid density can be selected via the switch 412 for the fluid selector 410. At block 612, the load cell voltage is measured. The load cell voltage can correspond with the mass of the IV bag 20 that contains the irrigating fluid 22. Accordingly, at block 614, the voltage is converted to mass. At block 616, the infused volume of the irrigating fluid 22 infused in the bladder of the patient during the cystoscopy procedure is calculated. The infused volume can be calculated by converting the mass of the IV bag 20 to volume of irrigating fluid 22 in the IV bag 20. The calculation of the volume of irrigating fluid 22 in the IV bag 20 can be determined as the density of the irrigating fluid 22 has been selected. The difference from the initial volume of irrigating fluid 22 in the IV bag 20 to the current volume and/or changed volume of the irrigating fluid 22 in the IV bag 20 is the infused volume in the bladder of the patient.

At block 626, if the reset button 166 is pushed, the process returns to block 602, where the calculation for infused volume is reset.

At block 618, the infused volume is displayed, for example by the user interface component 160 of the cystoscopy device. At block 620, to continue, the process proceeds to block 626 to determine if the reset button 166 is pushed. To not continue, the process proceeds to block 622 where the power switch 402 is actuated to end the procedure at block 624 and turn the cystoscopy device 100 off.

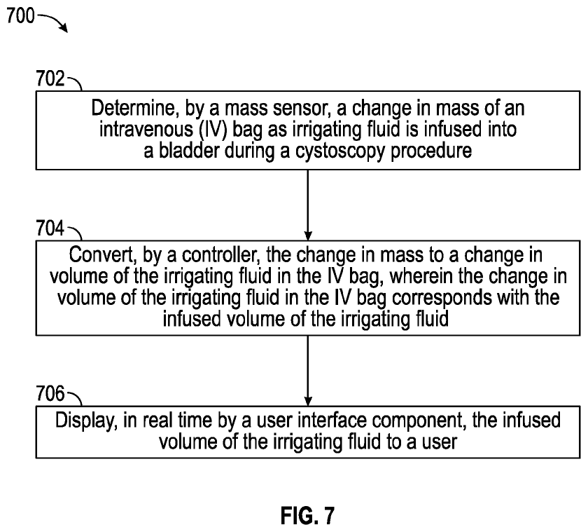

Referring to FIG. 7, a flowchart is presented in accordance with an example embodiment. The method 700 is provided by way of example, as there are a variety of ways to carry out the method. The method 700 described below can be carried out using the configurations illustrated in FIG. 1-6, for example, and various elements of these figures are referenced in explaining example method 700. Each block shown in FIG. 7 represents one or more processes, methods or subroutines, carried out in the example method 700. Furthermore, the illustrated order of blocks is illustrative only and the order of the blocks can change according to the present disclosure. Additional blocks may be added or fewer blocks may be utilized, without departing from this disclosure. The example method 700 can begin at block 702.

At block 702, a mass sensor 112 determines a change in mass of an intravenous (IV) bag 20 as irrigating fluid 22 is infused into a bladder during a cystoscopy procedure.

At block 704, a controller 500 converts the change in mass to a change in volume of the irrigating fluid 22 in the IV bag 20.

At block 706, a user interface component 160 displays in real time the infused volume of the irrigating fluid 22 to a user.

Having described several embodiments, it will be recognized by those skilled in the art that various modifications, alternative constructions, and equivalents may be used without departing from the spirit of the present inventive concept. Additionally, a number of well-known processes and elements have not been described in order to avoid unnecessarily obscuring the present inventive concept. Accordingly, this description should not be taken as limiting the scope of the present inventive concept.

Those skilled in the art will appreciate that the presently disclosed embodiments teach by way of example and not by limitation. Therefore, the matter contained in this description or shown in the accompanying drawings should be interpreted as illustrative and not in a limiting sense. The following claims are intended to cover all generic and specific features described herein, as well as all statements of the scope of the method and assemblies, which, as a matter of language, might be said to fall therebetween.

What is claimed is:

1. A cystoscopy device comprising:
a volume measurement component operable to determine in real time an infused volume of an irrigating fluid from an intravenous (IV) bag that is infused during a cystoscopy procedure; and
a user interface component in communication with the volume measurement component, the user interface component operable to display the infused volume of the irrigating fluid to a user.

2. The cystoscopy device of claim 1, wherein the volume measurement component includes a mass sensor operable to determine a mass of the IV bag.

3. The cystoscopy device of claim 2, wherein the mass sensor includes a load cell, wherein the IV bag is suspended from the load cell.

4. The cystoscopy device of claim 2, further comprising:
a controller coupled with the mass sensor, the controller operable to determine a change in mass of the IV bag and convert the change in mass to a change in volume of the irrigating fluid in the IV bag, the change in volume of the irrigating fluid in the IV bag corresponding with the infused volume of the irrigating fluid.

5. The cystoscopy device of claim 1, wherein the cystoscopy device is configured to be retrofit to a conventional cystoscope and a conventional IV bag.

6. The cystoscopy device of claim 1,
wherein,
the volume measurement component includes a mass sensor operable to determine a change in mass of the IV bag, and a controller coupled with the mass sensor,
the controller is operable to convert the change in mass to a change in volume of the irrigating fluid in the IV bag, and
the change in volume of the irrigating fluid in the IV bag corresponds with the infused volume of the irrigating fluid.

7. The cystoscopy device of claim 1, wherein the infused volume is displayed in real time.

8. The cystoscopy device of claim 1, wherein the user interface component is sterilizable.

9. The cystoscopy device of claim 1, wherein the user interface component is operable to be coupled with IV tubing that fluidly couples the IV bag with a cystoscope.

10. The cystoscopy device of claim 1, wherein the volume measurement component and/or the user interface component is powered by a battery.

11. A cystoscopy system comprising:
a cystoscope;
an intravenous (IV) bag containing irrigating fluid, the IV bag fluidly coupled with the cystoscope via an IV tubing such that the irrigating fluid is infused inside a bladder of a patient during a cystoscopy procedure; and
a cystoscopy device including:
a volume measurement component operable to determine in real time an infused volume of the irrigating fluid from the IV bag that is infused during the cystoscopy procedure; and
a user interface component in communication with the volume measurement component, the user interface component operable to display the infused volume of the irrigating fluid to a user.

12. The cystoscopy system of claim 11, wherein the volume measurement component includes a mass sensor operable to determine a mass of the IV bag.

13. The cystoscopy system of claim 12, wherein the mass sensor includes a load cell, wherein the IV bag is suspended from the load cell.

14. The cystoscopy system of claim 12, further comprising:
a controller coupled with the mass sensor, the controller operable to determine a change in mass of the IV bag and convert the change in mass to a change in volume of the irrigating fluid in the IV bag, the change in volume of the irrigating fluid in the IV bag corresponding with the infused volume of the irrigating fluid.

15. The cystoscopy system of claim 11, wherein the cystoscope is a conventional cystoscope and the IV bag is a conventional IV bag, wherein the cystoscopy device is configured to be retrofit to the conventional cystoscope and the conventional IV bag.

16. The cystoscopy system of claim 11,
wherein,
the volume measurement component includes a mass sensor operable to determine a change in mass of the IV bag, and a controller coupled with the mass sensor,
the controller is operable to convert the change in mass to a change in volume of the irrigating fluid in the IV bag, and
the change in volume of the irrigating fluid in the IV bag corresponds with the infused volume of the irrigating fluid.

17. The cystoscopy system of claim 11, wherein the infused volume is displayed in real time.

18. The cystoscopy system of claim 11, wherein the user interface component is sterilizable.

19. The cystoscopy system of claim 11, wherein the user interface component is operable to be coupled with the IV tubing.

20. A method comprising:
determining, by a mass sensor, a change in mass of an intravenous (IV) bag as irrigating fluid is infused into a bladder during a cystoscopy procedure;
converting, by a controller, the change in mass to a change in volume of the irrigating fluid in the IV bag, wherein the change in volume of the irrigating fluid in the IV bag corresponds with the infused volume of the irrigating fluid;
displaying, in real time by a user interface component, the infused volume of the irrigating fluid to a user.
